(19) United States
(12) Patent Application Publication
(10) Pub. No.: US 2014/0193326 A1
(43) Pub. Date: Jul. 10, 2014

(54) PROCESS FOR THE GASIFICATION OF WET BIOMASS

(71) Applicants: John Harinck, Delft (NL); Klaas Gerrit Smit, Anna Paulowna (NL)

(72) Inventors: John Harinck, Delft (NL); Klaas Gerrit Smit, Anna Paulowna (NL)

(73) Assignee: GENSOS HOLDING B.V., Breezand (NL)

(21) Appl. No.: 14/239,617

(22) PCT Filed: Aug. 20, 2012
(86) PCT No.: PCT/EP2012/066200

§ 371 Date: Feb. 19, 2014

(30) Foreign Application Priority Data
Aug. 26, 2011 (NL) 1039006

(51) Int. Cl. C07C 1/22; C01B 3/02

(52) U.S. Cl. CPC C07C 1/22; C01B 3/02
USPC 423/648.1; 585/240

(57) ABSTRACT

A process for the gasification of wet biomass comprises feeding the wet biomass at a temperature of at most 370 C. and a pressure of at least 22.1 MPa (absolute) to a reactor. The reactor comprises a bed of solid particles suspended in a fluid. The temperature of the feed is increased in the presence of the bed of suspended solid particles to a temperature of at least 375° C., forming supercritical water and converting in the presence of the supercritical water at least a portion of the organic materials present in the wet biomass into fluid gasification product.

The present invention relates to a process for the gasification of wet biomass.

Wet biomass, such as residues from fermentation facilities and animal manures, is available in vast quantities, and needs to be disposed of It comprises organic materials which can be converted in a high-temperature gasification reaction to a methane and hydrogen-rich gas. Methane and hydrogen are both valuable fuels. In this manner, wet biomass may in principle be an environmentally friendly and sustainable source of energy, which does not contribute to the build-up of greenhouse gasses in the atmosphere.

In addition to the organic materials, wet biomass comprises minerals, and other inorganic materials, such as sand and water. Water may be present in a substantial quantity. It has been suggested to perform the gasification of wet biomass at conditions at which the water is present in the reaction mixture as supercritical water. These conditions comprise a temperature which is above the critical temperature of water, which is 373.946° C., and a pressure which is above the critical pressure of water, which is 22.064 MPa (220.64 bar).

JP 2006021069-A (English language abstract) teaches that the gasification of wet biomass in the presence of supercritical water is highly efficient. However, there is a problem in that the solids formed in the process, such as salts, ash and char, tend to stick to the inner wall of the reactor and can cause clogging of the reactor. JP 2006021069-A (English language abstract) teaches a solution to this problem. In particular, JP 2006021069-A (English language abstract) teaches a process for the gasification of wet biomass in a reactor comprising a bed of solid particles suspended in a fluid, feeding the wet biomass comprising supercritical water at a temperature above the critical temperature of water to the reactor, and converting in the presence of the supercritical water at least a portion of the organic materials present in the wet biomass into fluid gasification product. In the process of JP 2006021069-A the reactor comprises a spouted bed.

The process of JP 2006021069-A may represent an improvement over the then existing prior art processes. However, it would appear that the problems of sticking and clogging have not been eliminated, and that these problems still exist in the heat exchanger in which the wet biomass is heated to a temperature above the critical temperature of water to form supercritical water, prior to feeding the wet biomass comprising supercritical water to the reactor.

It has now unexpectedly been found that these problems can be eliminated effectively by converting water present in the wet biomass into supercritical water in the presence of a bed of solid particles suspended in a fluid. Thus, according to the invention, the biomass is fed to the reactor at a temperature below the critical temperature of water, and, subsequently, inside the reactor comprising the bed of solid particles, the temperature of the feed is increased to the critical temperature or above, so that supercritical water is formed in the presence of the bed of solid particles.

The present invention therefore provides a process for the gasification of wet biomass, which process comprises

feeding the wet biomass at a temperature of at most 370° C. and a pressure of at least 22.1 MPa (absolute) to a reactor comprising a bed of solid particles suspended in a fluid,
increasing the temperature of the feed in the presence of the bed of suspended solid particles to a temperature of at least 375° C., forming supercritical water, and
converting in the presence of the supercritical water at least a portion of the organic materials present in the wet biomass into fluid gasification product.

The process of the invention may suitably be carried out in a reaction apparatus for the gasification of wet biomass, as described herein, which reaction apparatus comprises

a feeding system for feeding wet biomass at a pressure of at least 22.1 MPa (absolute),
a reactor comprising a reaction tube and a heating device, wherein
the reaction tube is fluidly connected to the feeding system,
the reaction tube is configured to comprise a bed of solid particles suspended in a fluid, and to receive wet biomass from the feeding system at a temperature below the critical temperature of water, and
the heating device is configured to heat the bed of suspended solid particles to a temperature above the critical temperature of water, and
   a recovery system, which recovery system is fluidly connected to the reaction tube for receiving fluid gasification reaction product from the reaction tube.

The invention provides the unexpected advantage over the existing processes that solids are formed under such circumstances that they do not stick to the inner wall of the equipment involved and that they do not cause clogging. Without wishing to be bound by theory, it is believed that solids are formed at the location where water present in the feed is converted into supercritical water, which—in the case of process of the invention—is in the reactor in which a bed of suspended solid particles is present. The presence of the bed of suspended solid particles prevents the solids formed from depositing on the inner wall of the equipment as a sticky material and causing clogging. The invention avoids supercritical water to be formed outside of the reactor.

As an additional advantage of the invention, the presence of solid particles improves the heat transfer from the heating device to the fluid at locations where there is a transition of subcritical water to supercritical water, or vice versa, in particular in case the fluid flow is in upward direction or in horizontal direction and the heat flux is high compared to the mass flux flow. Namely, without the solid particles being present, at such locations the heat transfer tends to deteriorate due to decreased turbulence caused by buoyancy effects. The deterioration of the heat transfer is diminished or neutralised by the presence of the solid particles.

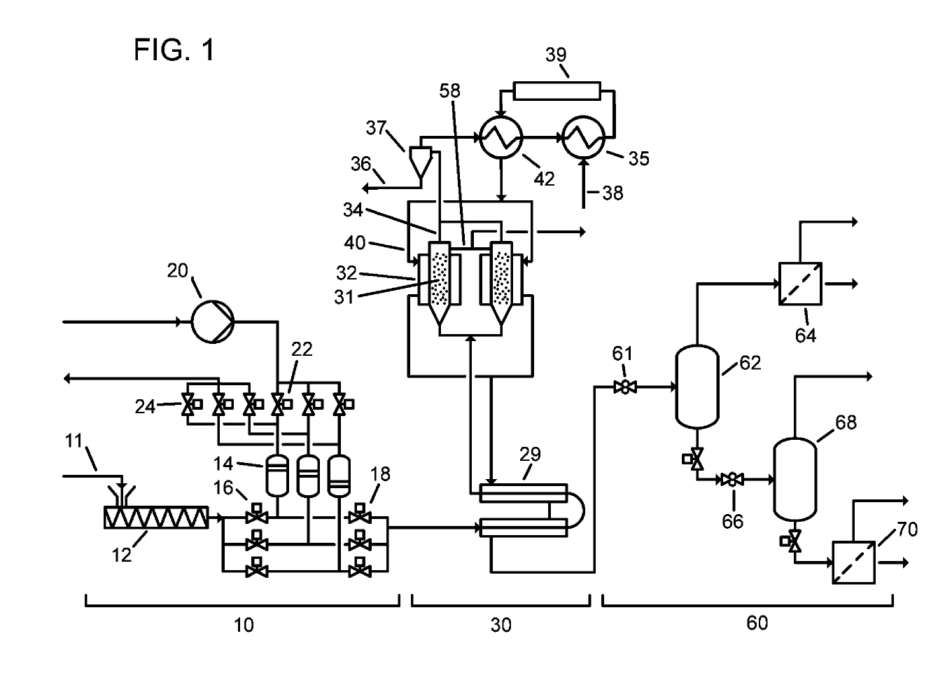

FIG. 1 provides a scheme of an embodiment of the process for the gasification of wet biomass in accordance with this invention and of a reaction apparatus, described herein, which is suitable for use in the process.

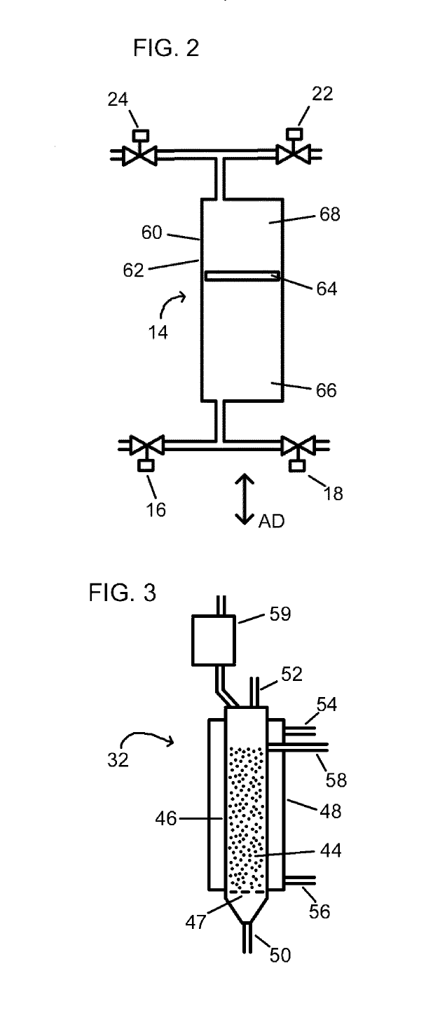

FIG. 2 provides a schematic of a portion of a feeding system for use in an embodiment of the gasification process in accordance with the invention.

FIG. 3 provides a schematic of a reactor which is suitable for use in an embodiment of the gasification process in accordance with this invention.

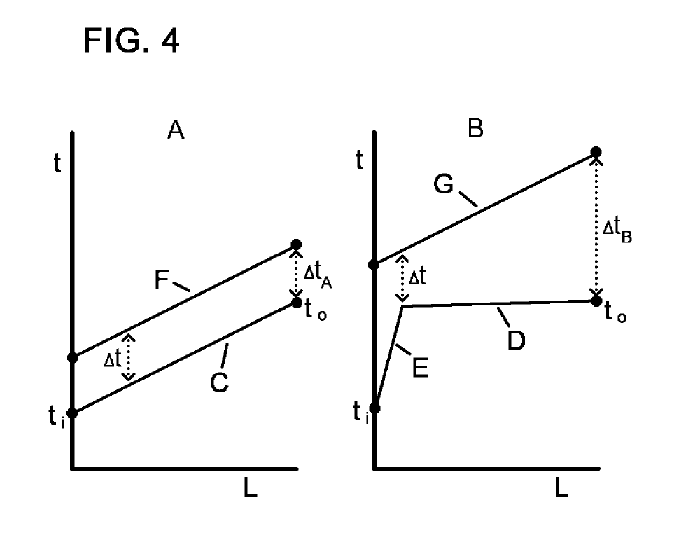

FIG. 4 shows temperature profiles of a reaction tube and a heating fluid over the length of the reaction tube with back-mixing and substantially without back-mixing in the reaction tube.

Throughout the Figures, the same objects will have the same reference numbers.

As used in this patent document, supercritical water is water above its critical temperature and above its critical pressure, and subcritical water is water below its critical temperature and above its critical pressure. It is generally known that water has its critical temperature at 373.946° C. and its critical pressure at 220.64 bar (22.064 MPa), cf. W. Wagner and A. Pruss, “The IAPWS Formulation 1995 for the Thermodynamic Properties of Ordinary Water Substance for General and Scientific Use,” J. Phys. Chem. Ref. Data, 31(2):387-535, 2002. As specified herein, pressure is absolute pressure. The term “fluid gasification product” is used herein in order to distinguish fluid (including gaseous and liquid) products of the gasification reaction from solid products, such as tars en solidified salts.

The wet biomass for use in the gasification process may be of various origins. The wet biomass may be, for example, residue from a fermentation facility, sewage sludge, dredging sludge, algae, or animal manures. Mixtures of wet biomasses of different origins may be employed. The wet biomass may or may not be pretreated before being introduced into the gasification process. Pretreating may involve shredding or cutting, for example, reducing the size or length of fibrous materials in the wet biomass, such as grass, straw or small stems. Water may be added to the wet biomass or water may be removed from the wet biomass, for example, to achieve a desired viscosity or density. Water may be removed by centrifuging or by gravitational sedimentation. Materials may be added to the biomass. For example, solid particles may be added to the wet biomass, supplementing solid particles of the bed of solid particles present in the reactor.

The wet biomass as fed to the gasification process comprises water, for example in a quantity of at least 40% w, typically at least 50% w, more typically at least 70% w, relative to the total weight of the wet biomass. In the normal practice of this invention, the water content is at most 95% w, on the same basis. The content of organic material is typically at least 1% w, more typically at least 5% w, and typically at most 60% w, more typically at most 50% w, on the same basis. The content of inorganic materials, other than water, is typically at least 1% w, more typically at least 3% w, and typically at most 80% w, more typically at most 60% w, on the same basis. The contents of organic and inorganic materials are as determined by thermal gravimetric analysis (TGA) in accordance with ASTM E1131-08.

The gasification process employs supercritical water which is formed in the reactor in the presence of the bed of solid particles. For this reason, the wet biomass is fed to the reactor at or above the critical pressure of water. The wet biomass is typically fed at a pressure of at least 22.5 MPa, preferably at least 23 MPa, more preferably at least 25 MPa. The pressure is typically at most 50 MPa, more typically at most 35 MPa, preferably at most 32 MPa, more preferably at most 30 MPa.

Wet biomass may be pressurised and fed to the reactor by using a pumping system. Eligible pumping systems may comprise a conventional high pressure pump, for example a piston pump or a membrane pump. However, such conventional pumping systems may be expensive as they must be robust and resistant to the action of fibrous material, sand and other solid particles, which may be present in the wet biomass and cause abrasion and/or clogging.

It has been found advantageous to employ a feeding pump for pumping wet biomass at low pressure into a cylinder. The cylinder comprises a piston which is movable in the axial direction of the cylinder. The piston, together with the cylinder walls, form two chambers inside the cylinder, which chambers are separated from each other by the piston. When wet biomass is fed at low pressure into the first chamber of the cylinder, and the first chamber receives wet biomass, the piston may move in the axial direction of the cylinder, away from the point of feeding wet biomass, so that the volume of the first chamber is increased. As used herein, the term “low pressure” may mean a pressure of less than 5 MPa. A suitable low pressure may be in the range of from 0.15 MPa to 5 MPa, more suitable in the range of from 0.2 MPa to 4 MPa, in particular in the range of from 0.3 MPa to 3 MPa. When subsequently a sufficiently high force is exerted onto the piston, which causes the piston to move into the opposite direction, the volume of the first chamber is decreased and wet biomass is discharged from the first cylinder at high pressure. Wet biomass so discharged at high pressure may be employed as feed in the gasification process of this invention. As used herein, the term “high pressure” may mean a pressure of at least 5 MPa, more typically at least 10 MPa, in particular at least 15 MPa, more in particular at least 20 MPa. The skilled person will appreciate that the force exerted onto the piston will be high enough to accommodate the pressure at which wet biomass is fed to the reactor, as specified hereinbefore.

Biomass may be fed to the first chamber by using a pump which operates at low pressure and which may be fluidly connected to the first chamber. Suitable pumps may be, for example, a worm pump or a lobe pump. The feeding pump may be equipped at the input side or at the output side with a shredder or cutter for reducing the size of fibrous material which may be present in the wet biomass.

The force which may be exerted onto the piston may be a mechanical force, using a screw or a piston rod. The force is preferably a hydraulic force exerted onto the piston by using a hydraulic fluid. The hydraulic fluid may be a hydraulic oil, but it is preferred to selected an aqueous liquid as the hydraulic fluid. The aqueous liquid may be filtered surface water, for example obtained from a river, a canal or a lake; or it may be tap water, drinking water, desalted water, or distilled water. Preferably, the aqueous liquid is filtered water.

The hydraulic fluid may be fed to the second chamber at high pressure by using a hydraulic pump which may be fluidly connected to the second chamber. For example, the pump may be a positive displacement pump, such as a piston pump, which may also be referred to as a plunger pump, or a membrane pump. When wet biomass is fed into the first chamber, and the piston moves in the axial direction of the cylinder, such that the volume of the first chamber increases, the volume of the second chamber decreases, with concomitant discharge of hydraulic fluid from the second chamber, for example into a reservoir which may also be used to hold a supply of hydraulic fluid as feed for the hydraulic pump. The skilled person will appreciate that the pressure at which the hydraulic fluid may be fed to the second chamber is equal to or higher than the high pressure, typically at most 2 MPa, in particular at most 1 MPa, more in particular at most 0.5 MPa, higher than the high pressure. The pressure at which the hydraulic fluid may be fed to the second chamber may typically be at least 0.001 MPa, in particular at least 0.01 MPa, higher than the high pressure.

A plurality of the cylinders comprising the piston, for example two, three or four cylinders with piston, may be employed in a parallel arrangement. By employing such an arrangement, a higher total feeding rate and/or an uninterrupted or continuous feed may be achieved. The skilled person will appreciate that the feeding system as described may employ valves which ensure that at any time the various streams of wet biomass and hydraulic fluid, if present, come from the appropriate source and find the appropriate destination. This will be set out further in the discussion of FIGS. 1 and 2, hereinafter.

The wet biomass fed to the reactor comprises water, which is typically subcritical water. The wet biomass may be preheated before feeding to the reactor. Typically, the wet biomass may be preheated to, and fed to the reactor at, a temperature of at most 360° C., more typically at most 350° C. Typically, the wet biomass may be preheated to, and fed to the reactor at, a temperature of at least 250° C., more typically at least 280° C., preferably at least 300° C.

In the reactor and in the presence of the bed of solid particles, the temperature of the wet biomass is increased to a temperature of at least 375° C., to the effect that supercritical water is formed from the water present in the wet biomass. Typically the temperature of the feed is increased to a temperature of at least 380° C., more typically 400° C., in particular at least 420° C. Typically the temperature of the feed is increased to a temperature of at most 800° C., more typically at most 760° C. The temperature of at least 375° C. may be selected such that the gasification reactions proceed at a rate as desired.

The bed of solid particles suspended in a fluid may preferably be a fluidised bed, typically a spouted fluidised bed or a circulating fluidised bed, and preferably a bubbling fluidised bed. In alternative embodiments the bed may be a fixed bed.

The fluid in which the solid particles are suspended is typically an aqueous fluid. Depending on the location in the reactor, the aqueous fluid may comprise supercritical water or subcritical water. Namely, close to a point of feeding the wet biomass, the temperature may be below the critical temperature of water, and at other points the temperature may be above the critical temperature of water. As gasification proceeds in the reactor, the fluid may also comprise fluid gasification products, in particular at locations away from the point of feeding the wet biomass.

The solid particles suspended in the fluid may be particles comprising, for example, a mineral or an aggregate of minerals, such as sand, crushed rock or crushed stone; a salt, for example a salt originating from wet biomass; metal, such as stainless steel, copper or aluminum; or a crystalline or non-crystalline ceramic, such as a glass, a clay, an alumina, a silica, a silica-alumina, or mixtures thereof. The material of the solid particles may have a density in a wide range, for example, in the range of from 1.5×10³ kg/m³ to 10×10³ kg/m³, more typically in the range of from 2×10³ kg/m³ to 9×10³ kg/m³. The particles may typically comprise particles having a size in the range of from 20 μm to 1 mm, in particular in the range of from 50 μm to 0.5 mm, wherein the size of the particles is as determined by ISO 13320:2009. Preferably, all particles have a size in the range as specified. The suspended solid particles may have a dual function in the gasification process, in that they assist in preventing solids from depositing on the inner wall of the reactor, and in addition they may act as a catalyst in the gasification reaction.

The solid particles may be fed into the reactor together with the wet biomass. For example, at least a portion of the solid particles may be sand which may inevitably be present in the wet biomass as one of its components. Alternatively, solid particles may be added to the wet biomass before feeding the wet biomass to the reactor. Dissolved salts which are present in the wet biomass may solidify in the reactor upon and/or after the formation of supercritical water, and such solidified salts may then constitute a portion of the bed of suspended solid particles. As another alternative, solid particles may be introduced into the reactor separate from the wet biomass.

The bed of suspended particles may have a void fraction which is selected from a wide range. Typically, the void fraction of the fluidised bed is in the range of from 0.05 to 0.95 v/v, relative to the total volume of the bed. When the bed is a bubbling fluidised bed, the void fraction may typically be in the range of from 0.25 to 0.8 v/v, more typically in the range of from 0.35 to 0.7 v/v, relative to the total volume of the bed. When the bed is a spouted fluidised bed, the void fraction may typically be in the range of from 0.05 to 0.2 v/v, relative to the total volume of the bed. When the bed is a circulating fluidised bed, the void fraction may typically be in the range of from 0.8 to 0.95 v/v, relative to the total volume of the bed. As used herein, the total volume of the bed is the volume of the bed at the conditions of temperature and pressure of the bed, and is as determined from the reactor dimensions and/or the dimensions of the portion of the reactor which holds the bed. The void volume is as determined by subtracting the particles volume from the bed volume. The particles volume may be as determined by submersing the particles present in the bed in water and determining the displaced volume of water.

The size of the reactor is not essential to the invention. Preferably, the residence time in the reactor is high enough for obtaining a sufficient yield of fluid gasification products. Thus, when the gasification process is operated in a continuous mode, the dimensions of the reactor are preferably such that at a desired throughput a sufficiently long residence time is achieved. It is also desired, for avoidance of the formation of tars, that in the reactor, or in the portion of the reactor which holds the bed of solid particles, the rate of temperature increase of the feed is high. Typically, the rate of temperature increase is at least 1.5° C./s, preferably at least 2° C./s. In the normal practice of the invention, the rate of temperature increase will frequently be at most 80° C./s, more frequently at most 50° C./s. The rate of temperature increase is as determined by calculating the quotient of the temperature increase and the average residence time of the fluid in the reactor or in the portion of the reactor which holds the bed of solid particles. The average residence time is determined from experiments using a tracer material.

The fluid gasification product may be withdrawn from the reactor together with supercritical water formed in the reactor. The fluid gasification product may also comprise entrained solid particles. A portion of the solid particles may remain in the reactor. Solid particles entrained in the fluid gasification product leaving the reactor may be removed. The fluid gasification product may be cooled and depressurised, resulting in a gas/aqueous liquid mixture, and gaseous gasification products may subsequently be recovered from the gas/aqueous liquid mixture.

In a preferred embodiment, prior to cooling, the fluid gasification product withdrawn from the reactor may be further heated. The further heated fluid gasification product may be used as a heating fluid (“first heating fluid”, hereinafter) for heating the reactor. It is generally sufficient to further heat fluid gasification product as to increase its temperature typically by at most 200° C., more typically by at most 150° C., for example 100° C. The temperature increase is typically at least 10° C., more typically at least 20° C. Electrical energy may be applied to accomplish the further heating. Preferably, the fluid gasification product withdrawn from the reactor is further heated by heat exchange with a second heating fluid. The second heating fluid may be a hot gas produced in a hot-gas producing unit. The hot-gas producing unit may be, for example, a gas burner, a gas turbine, a gas engine or a fuel cell.

For optimisation purposes, the further heated fluid gasification product may be kept at a high temperature for some time before the further heated fluid gasification product is used as the first heating fluid, as this will have the advantageous effect of increasing the methane content of the fluid gasification product. In this embodiment, the process may comprise as an additional step maintaining the temperature of the further heated fluid gasification product, typically for a period of at least 5 minutes, in particular at least 10 minutes, and typically for a period of at most 1 hour, in particular at most 40 minutes. This may be accomplished by using a vessel, preferably an insulated vessel or a heated vessel, which may hold the further heated fluid gasification for the time as specified. Herein, “maintaining the temperature” means maintaining the temperature typically within a margin of plus or minus 50° C., more typically within a margin of plus or minus 40° C., in particular within a margin of plus or minus 30° C.

In preferred embodiments, the further heated fluid gasification product may be used for the purpose of heating, by heat exchange, the wet biomass. In these embodiments, the temperatures of the wet biomass and the further heated fluid gasification product may be selected in accordance with the prevailing pressures, to the effect that an unexpected improvement in the heat integration of the gasification process is achieved. In the improved heat integration, the relatively large amount of heat released around the critical temperature when cooling down gasification product comprising supercritical water is used to satisfy the relatively large heat requirement around the critical temperature when heating wet biomass.

Accordingly, in these embodiments the gasification process comprises

heating wet biomass at a pressure Pₚ in the range of from 22.1 MPa to 35 MPa from a temperature of at most T₁ to a temperature of at least T₂ by heat exchange with a first heating fluid, upon which heating the fluid gasification product is obtained,
further heating the fluid gasification product, and
using the further heated fluid gasification product as the first heating fluid, upon which use the further heated fluid gasification product is cooled down at a pressure Pₛ in the range of from 22.1 MPa to 35 MPa from a temperature of at least T₃ to a temperature of at most T₄, wherein T₁, T₂, T₃ and T₄ are temperatures in ° C. which can be calculated by using the mathematical formulae

T₁=3.2×Pₚ+301.6,

T₂=3.8×Pₚ+292.4,

T₃=3.8×Pₛ+292.4, and

T₄=3.2×Pₛ+301.6,

wherein Pₚ and Pₛ denote the pressures Pₚ and Pₛ, respectively, in MPa. Preferably, T₁, T₂, T₃ and T₄ are temperatures in ° C. which can be calculated by using the mathematical formulae

T₁=2.9×Pₚ+306.2,

T₂=4.1×Pₚ+287.8,

T₃=4.1×Pₛ+287.8, and

T₄=2.9×Pₛ+306.2,

wherein Pₚ and Pₛ denote the pressures Pₚ and Pₛ, respectively, in MPa having values in the range of from 22.1 MPa to 33 MPa. More preferably, T₁, T₂, T₃ and T₄ are temperatures in ° C. which can be calculated using the mathematical formulae

T₁=2.6×Pₚ+310.8,

T₂=4.4×Pₚ+283.2,

T₃=4.4×Pₛ+283.2, and

T₄=2.6×Pₛ+310.8,

wherein Pₚ and Pₛ denote the pressures Pₚ and Pₛ, respectively, in MPa having values in the range of from 22.1 MPa to 32 MPa.

Typically the increase from the temperature of at most T₁ to the temperature of at least T₂ is at least 10° C., more typically at least 20° C., in particular at least 30° C. Typically the increase from the temperature of at most T₁ to the temperature of at least T₂ is at most 450° C., more typically at most 400° C., in particular at most 350° C. Typically the temperature of the feed is increased to a temperature of at least T₂ of at least 377° C., more typically at least 380° C., in particular at least 400° C., more in particular at least 420° C. Typically the temperature of the feed is increased to a temperature of at least T₂ of at most 800° C., more typically at most 760° C. At the temperature of at least T₂, as defined herein, water is present in the wet biomass as supercritical water. The temperature of at least T₃ may typically be at least 425° C., in particular at least 440° C., and typically at most 900° C., more typically at most 850° C. The decrease from the temperature of at least T₃ to the temperature of at most T₄ typically amounts to at least 10° C., more typically at least 20° C., in particular at least 30° C. Typically the decrease from the temperature of at least T₃ to the temperature of at most T₄ is at most 450° C., more typically at most 400° C., in particular at most 350° C. The further heated gasification product may be cooled down typically to a temperature of at most 390° C., in particular at most 380° C., more in particular at most 370° C., or even at most 360° C. Typically, it may be cooled down to a temperature of at least 300° C., more typically at least 320° C.

When, as in preferred embodiments, the gasification process is carried out as a continuous process, cooling down gasification product proceeds downstream from heating the wet biomass, in which case the pressure Pₛ is generally lower than the pressure Pₚ. Typically, the pressure Pₛ is at least 0.001 MPa, more typically at least 0.01 MPa, lower than the pressure Pₚ. Typically, the pressure Pₛ is at most 1 MPa, more typically at most 0.8 MPa, in particular at most 0.5 MPa, lower than the pressure Pₚ.

In an embodiment, the heat exchange may comprise heat exchange between a flow of the wet biomass and a flow of the further heated fluid gasification product which is co-current with the flow of the wet biomass. In such an embodiment, the temperature of at least T₃ and the temperature of at most T₄ are preferably both selected higher than the temperature of at least T₂. In a preferred embodiment, the heat exchange comprises heat exchange between a flow of the wet biomass and a flow of the further heated fluid gasification product which is counter-current with the flow of the wet biomass. The latter embodiment is preferred as the temperature of at least T₃ may be selected higher than the temperature of at least T₂ and the temperature of at most T₄ may be selected higher than the temperature of at most T₁, which makes the latter embodiment more energy efficient that the primer embodiment.

Now turning to the Figures, FIG. 1 provides a scheme of an embodiment of the process for the gasification of wet biomass in accordance with this invention and of a reaction apparatus, described herein, which is suitable for use in the process. The reaction apparatus may comprise feeding system 10, heating and reaction system 30 and recovery system 60.

Wet biomass 11 may be pressurised and introduced into heating and reaction system 30 by using a pumping system. It has been found advantageous to employ feeding pump 12 for pumping a portion of wet biomass 11 at low pressure into cylinder with piston 14, via valve 16. As an alternative to the use of feeding pump 12, wet biomass may be fed hydrostatically from a storage tank. Subsequently, valve 16 may be closed. Then, the wet biomass may be discharged at high pressure from cylinder with piston 14 via valve 18 into heating and reaction system 30, by using a hydraulic system comprising hydraulic pump 20 and valves 22 and 24. Hydraulic pump 20 may pump a hydraulic fluid via valve 22 into the second chamber of cylinder with piston 14, valves 16 and 24 being closed. After discharging the wet biomass into heating and reaction system 30, valve 18 may be closed, valves 16 and 24 may be opened and a further portion of wet biomass may be pumped from feeding pump 12 into cylinder 14. A plurality of cylinders with pistons 14 and a plurality of valves 16, 18, 22 and 24 may be placed in parallel arrangement.

FIG. 2 shows cylinder with piston 14, comprising cylinder wall 60. Piston 64 is located inside cylinder 62, and is movable in the axial direction AD of cylinder 62. Piston 64 divides the space inside cylinder 62 into first chamber 66 and second chamber 68. Piston 64 may be oriented generally perpendicularly relative to axial direction AD. Conduits may fluidly connect first chamber 66 via valve 16 to feeding pump 12 (FIG. 1) and via valve 18 to heating and reaction system 30 (FIG. 1). In addition, conduits may fluidly connect second chamber 68 via valve 22 to hydraulic pump 20 (FIG. 1) and via valve 24 to an outlet (not drawn) for hydraulic fluid or to a reservoir (not drawn) for holding a supply of hydraulic fluid.

The shape and size of cylinder 62 are not essential to the invention, and may be selected in accordance with the pumping capacity desired. Cylinder 62 may typically be a circular cylinder. The internal cross sectional area of the cylinder may typically be in the range of from 80 mm² to 20 dm², in particular in the range of from 7 cm² to 3.2 dm². The stroke of piston 64 may typically be in the range of from 0.1 m to 3 m, in particular in the range of from 0.2 m to 2.5 m. The wall thickness of the cylinder may typically be in the range of from 1 mm to 10 cm, in particular in the range of from 1.5 mm to 2 cm. The thickness of piston 64 may typically be in the range of from 1 mm to 30 cm, in particular in the range of from 1 cm to 20 mm. Cylinder 62 and piston 64 may typically be made of cast iron or steel, or a combination thereof. Cylinder with piston 14 may typically operate at a frequency in the range of from 0.1 strokes/minute to 50 strokes/minute, in particular a frequency in the range of from 0.2 strokes/minute to 20 strokes/minute, in which one stroke is a complete movement of the piston, which includes a movement towards the point of feeding wet biomass and a movement away from the point of feeding wet biomass.

Now turning again to FIG. 1, in heating and reaction system 30, heat exchanger 29 may be, for example, a double tube heat exchanger or a shell and tube heat exchanger. In heat exchanger 29, the wet biomass may be preheated to a temperature below the critical temperature of water, as set out hereinbefore. Then the preheated wet biomass may be introduced into reactor 32 comprising bed 31 of solid particles suspended in a fluid. In reactor 32 the wet biomass may be further heated to a temperature above the critical point of water, as set out hereinbefore. For the purpose of heating the wet biomass, reactor 32 comprises a heating device, for example a heating jacket and/or internal heating pipes through which a heating fluid may flow. A plurality of reactors 32 may be employed in parallel, to increase the total capacity of the reaction system.

Stream 34 of fluid gasification product leaving reactor 32 may preferably be treated to remove entrained solids, mainly comprising solid salts. In this preferred embodiment, the reaction apparatus comprises additionally a separation unit, in particular a cyclone, a gravity separator, or a device comprising impactor plates, positioned in the fluid connection connecting the reaction tube with the heater, which separation unit is configured to remove entrained solids from the fluid gasification product. Suitably, the removal may be achieved by using cyclone 37. Solids 36 may be discharged from cyclone 37, for example, via a lock chamber (not drawn). Removing solids at this point has an advantage that less heat is required when the fluid gasification product is further heated in a next heating step, as described hereinafter.

The heating fluid for use in reactor 32 may be any heating fluid which is high enough in temperature for sufficient heating of the wet biomass in the reactor. The heating fluid may be hot gas produced in a hot-gas producing unit. However, it has been found particularly advantageous to further heat fluid gasification product in heat exchanger 35 using the heat of, for example, hot gas 38 produced in a hot-gas producing unit (not drawn), and to use the further heated fluid gasification product as first heating fluid 40 in reactor 32. The hot-gas producing unit may be, for example, a gas burner, a gas turbine, a gas engine or a fuel cell. For optimisation purposes, vessel 39, for example a tube or an arrangement of parallel tubes, may be incorporated receiving further heated fluid gasification product from heat exchanger 35. Vessel 39 provides that further heated fluid gasification product will have an increased residence time at the highest temperature prevailing in heating and reaction system 30, which will have the advantageous effect of increasing the methane content of the fluid gasification product. In this embodiment, the reaction apparatus comprises additionally a vessel fluidly connected to the heater to receive further heated fluid gasification product from the heater and fluidly connected to the heating device to feed the further heated fluid gasification product into the heating device for use as the first heating fluid, which vessel is configured to hold the further heated fluid gasification product for a period of time.

With or without vessel 39 installed, additional heat exchanger 42 may be incorporated, transferring heat from further heated fluid gasification product to fluid gasification product before the latter enters heat exchanger 35. Alternatively, vessel 39 may be incorporated in the fluid connection between heat exchanger 42 and heat exchanger 35. In these embodiments, the reaction apparatus comprises additionally a heat exchanger positioned in the fluid connection connecting the heater with the heating device, and in the fluid connection connecting the reaction tube and with the heater, which heat exchanger is configured to exchange heat between the further heated fluid gasification product and the fluid gasification product.

It has been found particularly advantageous to employ as reactor 32 a reactor as shown in FIG. 3. Reactor 32 shown in FIG. 3 may comprise reaction tube 46, distribution plate 47, and the heating device, for example heating jacket 48 and/or in internal heating pipes (not drawn). Inlet pipe 50 for wet biomass may be fluidly connected with heat exchanger 29 (FIG. 1). Outlet pipe 52 for fluid gasification product may be fluidly connected to heat exchanger 35, optionally via heat exchanger 42 and/or cyclone 37 and/or vessel 39. Inlet pipe 54 for heating fluid may be fluidly connected with heat exchanger 35, optionally via heat exchanger 42 and/or vessel 39. Outlet pipe 56 for heating fluid may be fluidly connected with heat exchanger 29. The bed of suspended solid particles in the form of a fluidised bed 44 may be contained in reaction tube 46, downstream of distribution plate 47. An excess of solid particles may be withdrawn from reactor 32 via overflow pipe 58. Reaction tube 46 is adapted to allow wet biomass to pass in the longitudinal direction of the reaction tube, and counter-currently with the heating fluid flowing in heating jacket 48 and/or in internal heating pipes. Reactor 32 may be fluidly connected with lock chamber 59 for the purpose of introducing solid particles into the reactor.

Fluidised bed 44 has typically a length of at least 0.5 m, more typically at least 1 m. Fluidised bed 44 has typically a length of at most 10 m, more typically at most 5 m. For example, the length of fluidised bed 44 may suitably be 3 m. The cross sectional area of fluidised bed 44 is typically at most 20 dm², more typically at most 5 dm² and most typically at most 2 dm². The cross sectional area of fluidised bed 44 is typically at least 1 cm², more typically at least 2 cm². For example, the cross sectional area of fluidised bed 44 may suitably be 4.5 cm². Preferably, fluidised bed 44 has the shape of a circular cylinder, typically having a length to diameter ratio in the range of from 5 to 50, more typically in the range of from 8 to 30. For example, the length to diameter ratio of fluidised bed 44 may suitably be 20. In fluidised bed 44, when having dimensions as specified in this paragraph, there is relatively little back-mixing, so that there is a temperature gradient over the length of the bed. A single reactor tube comprising a fluidised bed having dimensions as specified may be installed. Alternatively, a plurality of reaction tubes comprising a fluidised bed having dimensions as specified may be installed in parallel. The number of reaction tubes and fluidised beds may be in the range of from 2 to 20 (inclusive), in particular in the range of from 3 to 10 (inclusive).

An advantage of having fluidised bed 44 in which there is relatively little back-mixing is shown in FIG. 4. FIG. 4 shows the profiles of temperature t over length L of the bed and the heating fluid, substantially without back-mixing in the bed (situation A) and, for comparison, with substantial back-mixing in the bed (situation B). In situation A, there is a temperature gradient C in the bed, which extends from an inlet temperature tᵢ to an outlet temperature tₒ. In situation B, there is, as a consequence of back-mixing, virtually the same temperature over the length of the bed (D), except for a steep temperature gradient E near the inlet. F and G depict the temperature profiles of the heating fluids which can accommodate the heating profiles of the reaction tubes in situations A and B, respectively. In both cases Δt depicts the minimum temperature difference between the reaction tube and the heating fluid. With Δt being equal in situations A and B, temperature difference Δt_{A} at the inlet of the heating fluid in situation A is substantially less than temperature difference Δt_{B} at the inlet of the heating fluid inlet in situation B. This implies for the process depicted in FIG. 1 that in heat exchanger 35 fluid gasification product needs heating to achieve a smaller temperature increase in situation A than in situation B, which means that in situation A less heat is supplied from the hot-gas producing unit than in situation B.

Returning again to FIG. 1, further heated fluid gasification product may be used as first heating fluid 40 in heating jacket 48 and/or in internal heating pipes (not drawn) of reactor 32. When the fluid gasification product leaves reactor 32 through outlet pipe 56 it may typically have a temperature in the range of from 300° C. to 450° C., more typically in the range of from 350° C. to 400° C., for example 380° C. The fluid gasification product may be cooled down further in heat exchanger 29 against wet biomass. The fluid gasification product leaving heat exchanger 29 may typically have a temperature in the range of from 20° C. to 150° C., more typically in the range of from 40° C. to 120° C., for example 90° C.

The extensive recovery of heat as it may take place in any of heat exchangers 42 and 29 and in reactor 32 renders the process depicted in FIG. 1 a very energy efficient process in that the recovery of heat reduces the net energy supply to the process, in particular the energy supply in heat exchanger 35 by means of the hot gas 38.

When entering recovery system 60, the fluid gasification product may be depressurized over valve 61 to a pressure typically in the range of from 0.1 MPa to 20 MPa, more typically in the range of from 0.2 MPa to 15 MPa.

Depressurised fluid gasification product may be degassed in degasser 62, yielding a gas fraction and a liquid fraction. The gas fraction comprising high value gases, such as hydrogen and methane may be split into a methane-rich stream and a hydrogen-rich stream in, for example, membrane separator 64. The liquid fraction from degasser 62 may be depressurized further over valve 66, and further degassed in degasser 68, producing a gaseous fraction which may comprise carbon dioxide, methane, hydrogen, and hydrocarbons other than methane. The pressure downstream of valve 66 may typically be in the range of from 0.1 MPa to 5 MPa, more typically in the range of from 0.2 MPa to 3 MPa. The liquid product obtained in degasser 68 is an aqueous residue comprising salts. The aqueous residue may be treated in membrane separator 70 yielding water and an aqueous residue being enriched in salts.

1. A process for the gasification of wet biomass, which process comprises
feeding the wet biomass at a temperature of at most 370° C. and a pressure of at least 22.1 MPa (absolute) to a reactor comprising a bed of solid particles suspended in a fluid,
increasing the temperature of the feed in the presence of the bed of suspended solid particles to a temperature of at least 375° C., forming supercritical water, and
converting in the presence of the supercritical water at least a portion of the organic materials present in the wet biomass into fluid gasification product.

2. A process as claimed in claim 1, wherein the wet biomass is selected from fermentation residues, sewage sludge, dredging sludge, algae, animal manures, and mixtures thereof.

3. A process as claimed in claim 1 or 2, wherein the wet biomass comprises at least 40% w of water, relative to the total weight of the wet biomass.

4. A process as claimed in any of claims 1-3, wherein the wet biomass is fed at a pressure in the range of from 22.1 MPa to 50 MPa, in particular a pressure in the range of from 22.5 MPa to 35 MPa.

5. A process as claimed in any of claims 1-4, wherein the wet biomass is fed at a temperature in the range of from 280° C. to 360° C.

6. A process as claimed in claim 5, wherein the wet biomass is fed at a temperature in the range of from 300° C. to 350° C.

7. A process as claimed in any of claims 1-6, wherein the temperature of the feed is increased in the presence of the bed of suspended solid particles to a temperature in the range of from 400° C. to 800° C.

8. A process as claimed in claim 7, wherein the temperature of the feed is increased in the presence of the bed of suspended solid particles to a temperature in the range of from 420° C. to 760° C.

9. A process as claimed in any of claims 1-8, wherein the bed of suspended solid particles is a fluidised bed.

10. A process as claimed in claim 9, wherein the fluidised bed is a bubbling fluidised bed.

11. A process as claimed in any of claims 1-10, wherein the fluid is an aqueous fluid.

12. A process as claimed in any of claims 1-11, wherein the suspended solid particles comprise particles selected from minerals or aggregates of minerals, crushed rock or crushed stone, salts, metals, crystalline or non-crystalline ceramics, and mixtures thereof.

13. A process as claimed in any of claims 1-12, wherein the suspended solid particles comprise particles having a size of at least 20 μm, wherein the size of the particles is as determined by ISO 13320:2009.

14. A process as claimed in claim 13, wherein the suspended solid particles comprise particles having a size in the range of from 20 μm to 1 mm, wherein the size of the particles is as determined by ISO 13320:2009.

15. A process as claimed in claim 14, wherein the suspended solid particles comprise particles having a size in the range of from 50 μm to 0.5 mm, wherein the size of the particles is as determined by ISO 13320:2009.

16. A process as claimed in any of claims 1-15, wherein the rate of temperature increase is in the range of from 1.5° C./s to 80° C./s, in particular in the range of from 2° C./s to 50° C./s.
